(19) United States
(12) Patent Application Publication
(10) Pub. No.: US 2021/0022671 A1
(43) Pub. Date: Jan. 28, 2021

(54) ADAPTIVE DEPTH OF ANESTHESIA MONITOR

(71) Applicant: Oridion Medical 1987 Ltd., Jerusalem (IL)

(72) Inventors: Myriam Lesmy, Jerusalem (IL); Michal Ronen, Jerusalem (IL); Yoni Schwartz, Efrat (IL)

(21) Appl. No.: 16/937,251

(22) Filed: Jul. 23, 2020

Related U.S. Application Data
(60) Provisional application No. 62/879,217, filed on Jul. 26, 2019.

(51) Int. Cl. A61B 5/00; A61B 5/0476; A61B 5/0488; A61B 5/0496; A61B 5/0205; G16H 50/30; G16H 10/60; G16H 40/63; G16H 50/20

(52) U.S. Cl. CPC A61B 5/4821; A61B 5/0476; A61B 5/0488; A61B 5/0496; A61B 5/0205; A61B 5/021; G16H 50/30; G16H 10/60; G16H 40/63; G16H 50/20; A61B 5/742

(57) ABSTRACT

In some examples, a method including determining an effective brain age metric for a patient based on at least one brain signal of the patient; receiving a signal indicative of a physiological parameter of the patient; and generating an index score indicative of depth of anesthesia (DOA) of the patient based on the received signal and the determined effective brain age metric for the patient.

This application claims the benefit of U.S. Provisional Application No. 62/879,217 entitled ADAPTIVE DEPTH OF ANESTHESIA MONITOR, filed Jul. 26, 2019, the entirety of which is hereby incorporated by reference.

### TECHNICAL FIELD

The disclosure generally relates to patient monitoring.

### BACKGROUND

A patient undergoing a medical procedure may be anesthetized by receiving one or more pharmacological anesthetic agents. Different anesthetic agents may produce different effects, such as sedation or hypnosis (e.g., the lack of consciousness or awareness of the surrounding world), analgesia (e.g., the blunting or absence of pain), or paralysis (e.g., muscle relaxation, which may or may not result in lack of voluntary movement by the patient). Anesthetic agents may provide one or more of these effects and to varying extents on different patients. For example, neuromuscular blocking agents may provide potent paralysis, but no sedation or analgesia. Opioids may provide analgesia and relatively light levels of sedation. Volatile anesthetic agents may provide relatively significant levels of sedation and much smaller levels of analgesia, while the intravenous sedative agent propofol may provide sedation but essentially no analgesia. For this reason, anesthesia providers may simultaneously administer several of these agents to a patient to provide the desired set of effects. For example, an anesthesia provider may administer to a patient a volatile anesthetic agent for its sedative effect, a neuromuscular blocking agent for paralysis and an opioid agent to provide analgesia. In general, the magnitude of the effects provided by these agents are dose-dependent; the higher the dose, the more profound the effect.

### SUMMARY

The present disclosure describes devices, systems, and techniques for assessing a patient's depth of anesthesia (DOA) (also referred to as depth of consciousness in some examples) before, during, and/or after a medical procedure (e.g., a surgical procedure). For example, the systems and techniques may be used, e.g., by a clinician or other medical personnel, to evaluate a patient before or during a medical procedure (e.g., during which the patient is anesthetized for a period of time while a surgeon operates on the patient) to determine a DOA index score for the patient, which is indicative of a determined DOA for the patient, e.g., for a particular time or time period.

In examples described herein, processing circuitry of a medical device system is configured to generate a DOA index score for a patient based on at least one physiological parameter of the patient indicated by signals received by the processing circuitry. Example signals indicative of a patient physiological parameters that may be used to determine the DOA index score may include, but are not limited to, an electroencephalogram (EEG) signal, an electromyography (EMG) signal, an electrooculography (EOG) signal, a blood pressure (BP) signal, a heart rate (HR) signal, a temperature signal, a pulse oximeter (SpO₂) signal, a plethysmograph (finger and/or forehead) signal, a facial expression signal (e.g., as received from camera used to visually monitor the patient), a facial color signal (e.g., as received from camera used to visually monitor the patient), a capnogram signal, and/or an auditory evoked potentials (AEP).

The processing circuitry may be configured to determine an effective brain age metric of a patient, e.g., prior to the patient being anesthetized, as a part of the DOA monitoring process. The effective brain age of the patient may be different from the biological age of the patient, and may reflect, e.g., changes in the brain of a patient resulting from structural, chemical and functional changes while the patient has aged over time. Such changes may alter the EEG signal, other brain signals, and/or other physiological signals of the patient when anesthetized, including the characteristics of the signals that may be indicative of the relative level of DOA while the patient is anesthetized. The processing circuitry may adapt the DOA monitoring algorithm or other evaluation techniques based on the determined effective brain age of the patient, e.g., such that the processing circuitry more accurately generates an DOA index score based on monitored physiological parameters, and characteristics thereof, that reflect the relative level of DOA of the patient for a patient having the determined effective brain age.

In some examples, the disclosure is directed to a method comprising determining, via processing circuitry, an effective brain age metric for a patient based on two or more patient parameters; receiving a signal indicative of a physiological parameter of the patient; and generating, via the processing circuitry, an index score indicative of depth of anesthesia (DOA) of the patient based on the received signal and the determined effective brain age metric for the patient.

In some examples, the disclosure is directed to a system comprising processing circuitry configured to determine an effective brain age metric for a patient based on two or more of patient parameters; receive a signal indicative of a physiological parameter of the patient; and generate, via the processing circuitry, an index score indicative of depth of anesthesia (DOA) of the patient based on the received signal and the determined effective brain age metric for the patient.

In some examples, the disclosure is directed to a method comprising determining, via processing circuitry, an effective brain age metric for a patient based on at least one brain signal of the patient; receiving a signal indicative of a physiological parameter of the patient; and generating, via the processing circuitry, an index score indicative of depth of anesthesia (DOA) of the patient based on the received signal and the determined effective brain age metric for the patient.

In some examples, the disclosure is directed to a system comprising processing circuitry configured to determine an effective brain age metric for a patient based on two or more of patient parameters; receive a signal indicative of a physiological parameter of the patient; and generate, via the processing circuitry, an index score indicative of depth of anesthesia (DOA) of the patient based on the received signal and the determined effective brain age metric for the patient.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below

### BRIEF DESCRIPTION OF DRAWINGS

The above and other features of the present disclosure, its nature and various advantages will be more apparent upon consideration of the following detailed description, taken in conjunction with the accompanying drawings in which:

FIG. 1 is a schematic and conceptual diagram illustrating an example system for evaluating a patient's depth of anesthesia.

FIG. 2 is a schematic and conceptual diagram of a system for evaluating a patient's depth of anesthesia.

FIG. 3 is a flow diagram illustrating an example technique for determining a patient's depth of anesthesia.

FIG. 4 is a flow diagram illustrating another example technique for determining a patient's depth of anesthesia.

### DETAILED DESCRIPTION

In some examples, the disclosure describes systems, devices, and techniques for evaluating a patient's depth of anesthesia, before, during, and/or following a medical procedure (e.g., a surgical procedure during which the patient is operated on by a surgeon). For example, such an evaluation may be performed on the patient pre-operatively and/or during the medical procedure while the patient is anesthetized. The evaluation may be expressed as a depth of anesthesia (DOA) index score that reflects the relative DOA for a patient. As described herein, the evaluation may utilize a metric for determining the effective brain age of a patient (referred to herein as an effective brain age metric) such that the DOA evaluation may be adapted to the effective brain age of the patient (e.g., rather than using the same evaluation algorithm for all patient or adapting the evaluation algorithm based solely on the biological age of the patient).

DOA monitors, including the Bispectral Index™ (BIS) monitor available from Medtronic plc (Dublin, Ireland), may be used in an operating room (OR) to assist a clinician in controlling the anesthetic drugs dosage administered to a patient. For example, a bispectral (BIS) index may be a processed parameter which may be derived utilizing a composite of measures from EEG and physiological signal processing techniques including bispectral analysis, power spectral analysis, and time domain analysis. The BIS algorithm may be based at least in part on EEG signal features (bispectral and others) which may be highly correlated with sedation/hypnosis, including the degree of high frequency (14 to 30 Hz) activation, the amount of low frequency synchronization, the presence of nearly suppressed periods within the EEG, and the presence of fully suppressed (i.e., isoelectric, “flat line”) periods within an EEG. The BIS index may provide an indication of a subject's DOA, with an index value of 0 representing a “flat line” EEG and an index value of 100 indicating a fully awake subject. Such a DOA measure may be used by care providers in operating room or intensive care settings to evaluate a patient's status and provide treatment accordingly (e.g., adjusting anesthetic or analgesic administration).

The DOA determination may be helpful for avoiding various adverse reactions or situations, such as, but not limited to, intraoperative awareness with recall, prolonged recovery, and/or an increased risk of postoperative complications for a patient, such as post-operative delirium. Studies have shown that DOA monitoring using electroencephalography (EEG) may improve patient treatment and outcomes by reducing the incidences of intraoperative awareness, minimizing anesthetic drug consumption, and resulting in faster patient wake-up and recovery.

Monitoring DOA in elderly patients in terms of biological age may be particularly useful, e.g., since a sub-optimal titration of anesthetic drugs may increase the risk of complications following surgery, decrease the chances of complete recovery, and may have other adverse consequences. The aging brain encounters structural, chemical and functional changes altering the EEG signal, other brain signals, and/or other physiological parameters, and as a consequence, example DOA monitoring algorithms may be less accurate, e.g., when monitoring the elderly. In some example, the “brain age” may be directly affected not only by the biological age of the patient, but also by the lifestyle, comorbidities, mental state, and physical condition of the patient.

In other words, an elderly patient in terms of biological age can have a brain corresponding to an effective older or younger age. As an illustration, both a first patient and a second patient twenty years older than the first patient in terms of biological age may have an effective brain age that is substantially the same, e.g., in the context of structure, chemistry, and function despite the biological age difference. Likewise, for two patients of the same biological age, the brain signals of one of the patients may be different from that of the brain signals of the other patient despite being the same biological age. The differences may result from the two patient's brains undergoing different structural, chemical and functional changes during biological aging. As a result, a DOA monitoring system such as a BIS monitor that analyzes the brain signals of the patients using, e.g., the same algorithm, may more accurately determine one of the patient's DOA compared to the other patient's DOA despite the patient's being the same biological age.

In accordance with examples of the disclosure, devices, systems, and techniques are described which generate a DOA index score of a patient indicative of the relative DOA of a patient based in part on a determine effective brain age of the patient. For example, a monitoring system may employ analysis techniques when evaluating the brain signals (and/or other physiological parameters of the patient that are indicative of the patient's DOA) that are adapted based on the effective brain age determined for a patient. The patient's effective brain age may be determined at least in part during an evaluation period prior to the patient being anesthetized. Example parameters that may be used to determine the effective brain age of the patient may include, e.g., biological age of the patient, EEG signals and/or other brain signals of the patient when not anesthetized, co-morbidities of the patient, frailty of the patient, one or more other physiological parameters, such as, heart rate and/or blood pressure.

The monitoring algorithm employed to determine the patient's DOA when subsequently anesthetized may be selected based on the determined effective brain age for the patient. The selected monitoring algorithm may evaluate the brain signal(s) and other physiological parameters to identify characteristics that reflect the patient's actual DOA for the particular effective brain age of the patient. Those identifiable characteristics may be unique to the effective brain age of the patient and may not accurately reflect the actual DOA for a patient having a different effective brain age. In other words, characteristics of brain signals and/or other monitored parameters of a patient may be indicative of substantially the same relative level of DOA for patients having the same effective brain age while those same characteristics may not indicate that same relative level of DOA in patients having a different effective brain age. Thus, by determining the effective brain age of a patient, a DOA monitoring system may be adapted to more accurately determine the DOA of the patient based on the determined effective brain age, e.g., by identifying one or more characteristics indicative of particular level of DOA associated with the determined effective brain while the patient is anesthetized, e.g., even though the same identifiable characteristic may not indicate the same level of DOA for a patient having a different effective brain age.

In some examples, an adaptive DOA monitoring algorithm may be employed that determines the DOA for a patient by taking into account not only the EEG signal (and/or other brain signals of the patient) and the biological age of the patient, but also additional parameters of the patient such as the cognitive state using questionnaires and/or prior EEG recordings, physiological parameters such as BP, HR, etc, and the patient's medical history. These additional parameters may be important to assess the patient's condition and to establish accurately depth of anesthesia as well as an adapted drug titration.

The generated DOA index score may be a numerical value on a scale used to indicate the relative depth of anesthesia of a patient (e.g., a scale of 1 to 100, wherein an index score of 1 indicates a very low level or substantially no anesthesia of the patient and an index score of 100 indicates a very high level of anesthesia of the patient, or a scale of 1 to 10, or another numerical scale). In some examples, the treatment of the patient before, during, and/or after the medical procedure may be tailored based on the DOA index score. In this manner, the overall treatment of a patient undergoing surgery may be improved by, e.g., by modifying the anesthesia administered to a patient based on a determined DOA index score before, during, and/or after the medical procedure as desired.

In some examples, the disclosure describes an adaptive algorithm for assessing DOA, particularly in elderly patients. Prior to surgery, the cognitive and the physical states of a patient may be assessed by questionnaires or other techniques, and scored. In addition to the fluctuations of the brain activity recorded by, e.g., an EEG, the DOA monitoring algorithm may take into account parameters such as: biological age, co-morbidities, frailty, and baseline EEG recordings prior to surgery to properly compute an adaptive DOA index score, as well as physiological parameters such as blood pressure, heart rate and others. The algorithm may identify patterns or other characteristics in an EEG or other brain signal which are specific to aging (changes in beta, alpha, delta, gamma and theta bands, decreasing number of K-complexes and spindles, and others). For example, an initial step may be to assess the EBA of a patient. This may be done is some examples either by a clinician evaluating the patient by questionaire (such as frailty and mini-mental questionaires), and/or by only using an EEG signature (or other brain signal signature) taken prior to surgery. In some examples, the new algorithm uses state-of-the-art methods such as wavelet transforms, the bispectrum, blind source separation and neural networks, in order to calculate a more accurate depth of anesthesia index for the elderly. The algorithm may have a number of stages, where the brain signals are initially characterized and classified, and then the DOA index score is computed using an algorithm or other evaluation technique that is tailored for the specific population (e.g., a population corresponding to the same effective brain age). Such monitoring techniques may result in improved patient care, e.g., as a result of a determining a DOA index score that better reflects the actual DOA of a patient.

Example physiological parameter signals of the patient that may be used by the monitoring system to generate a DOA index score include, e.g., an electroencephalogram (EEG) signal, an electromyography (EMG) signal, an electrooculography (EOG) signal, a blood pressure (BP) signal, a heart rate (HR) signal, a temperature signal, a pulse oximeter (SpO₂) signal, a plethysmograph (e.g., generated based on a sensor at a finger and/or a forehead of the patient) signal, a facial expression signal (e.g., as received from camera used to monitor the patient), a facial color signal (e.g., as received from camera used to monitor the patient), a capnogram signal, and/or an auditory evoked potentials (AEP). Such parameters may be measured using devices which can be found in many operating rooms, or that may be relatively easy to integrate into the operating room setting. In some examples, the main signal used as an indicator of the EBA of the patient is the EEG. The other parameters listed above may be used in conjunction with the EEG to determine the DOA, e.g., regardless of the age.

The physiological parameters used by a monitoring system (also referred to as DOA assessment system in some examples) described herein to generate a DOA index score for a patient may have identifiable characteristics that reflect a relative level of DOA of the patient. The physiological signals may, for example, exhibit identifiable characteristics (e.g., patterns or other characteristics) that are indicative of the relative level of a patient's DOA. In some examples, the identifiable characteristics indicative of the relative level of DOA may be specific to a particular effective brain age or range of effective brain ages. Conversely, the same identifiable characteristic may be indicative a different relative level of DOA or may not be indicative of a relative level of DOA for a patient having a different effective brain age or range of effective brain age. By determining the effective brain age of a patient, a DOA monitoring system may be adapted to identify one or more characteristics indicative of a relative level of DOA for the determined effective brain age of the patient, and then generate a DOA index score for the patient based at least in part on the identifiable characteristics associated with the determined effective brain age. For example, example characteristics may be identified from one or more features derived from an EEG (or other brain signal) including, e.g., beta band to delta band ratio, bands peak location, which contribute differently per EBA. Thus, those and other features may have different weights when calculating the DOA index score for a patient based on the EBA.

In some examples, once processing circuitry of a monitoring system has determined the DOA index score for a patient, the monitoring system may display or otherwise report the determined DOA index score, e.g., to a clinician or other medical personnel. In some examples, the DOA index score may be display in terms of the numerical scale, e.g., on a scale of 1 to 100, where 1 indicates the no DOA or the lowest DOA and where 100 indicates the highest DOA for a patient. Alternatively, or additionally, the DOA index score for patient may be indicated via display of a non-numerical technique such as, e.g., using a color scale where different colors correspond to different relative levels of DOA (e.g., green reflecting a desired DOA, and red reflecting an undesirable DOA) or text stating the level of DOA (e.g., “low DOA,” “medium DOA,” or “high DOA”).

In some examples, for patients determined to have a relatively low or high DOA index score, the anesthesia management or protocol in an operating room setting, e.g., type of anesthesia (general, spine) type of drugs used, rate of titration during induction, monitoring of the patient's sedateness, and the like, for the patient may be modified to account for the relatively low or high DOA index score. For example, the DOA monitoring system be configured to provide a recommendation of a course of action to a clinician, e.g., to modify one or more particular parameters (e.g., drug delivery boluses of particular drugs) of anesthesia agents being delivered to the patient to improve the patient's DOA.

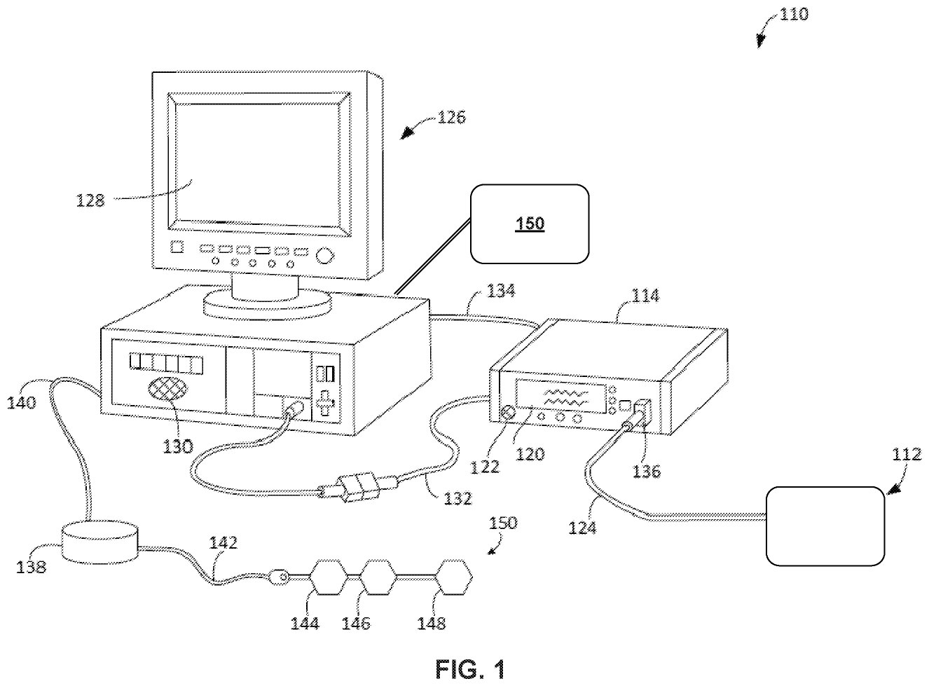

FIG. 1 is a perspective view of an example monitoring system 110 in accordance with some examples of the present disclosure. Monitoring system 110 may be a DOA assessment system in that monitoring system 110 may be configured to generate a DOA index score for a patient, e.g., before, during, and/or after a medical procedure. In some examples, monitoring system 110 may be implemented as part of an EEG, EOG, ECG, and/or EMG monitoring system. In some examples, monitoring system 110 may be implemented as part of a DOA system, such as, e.g., a monitoring system configured to generate a Bispectral Index (BIS®) including an example monitoring system of the type disclosed in U.S. Pat. No. 5,458,117, issued Oct. 17, 1995, the entirety of which is incorporated by reference herein.

In some examples, monitoring system 110 may include sensor unit 112 and monitor 114. In some examples, sensor unit 112 may include an oximeter sensor or other sensor configured to sense blood pressure, heart rate, and the like, or any combination thereof. Sensor unit 112 may include a light source configured to emit light at one or more wavelengths into a subject's tissue and a detector configured to detect the light that is reflected by or has traveled through the subject's tissue. Monitoring system 110 may also include one or more additional sensor units (not shown) that may, for example, take the form of any of the examples described herein with reference to sensor unit 112. An additional sensor unit may be the same type of sensor unit as sensor unit 112, or a different sensor unit type than sensor unit 112 (e.g., a photoacoustic sensor). Multiple sensor units may be capable of being positioned at two different locations on a subject's body. Sensor unit 112 may also be included in an array of one or more additional types of sensors (e.g., electrodes for sensing electrophysiological signals such as EEG, EMG, ECG, and/or EOG signals). For example, sensor unit 112 may be included in a multi-sensor array configured to be located on a subject's head. Additional examples are described in detail below.

In some examples, sensor unit 112 may be connected to monitor 114 as shown. In Sensor unit 112 may be powered by an internal power source, e.g., a battery (not shown), may draw power from monitor 114, or may be powered by another power source. In another example, sensor unit 112 may be wirelessly connected (not shown) to monitor 114. Monitor 114 may be configured to determine a DOA index score based at least in part on data received from any sensor of any type (e.g., an EEG, EOG, ECG, or EMG electrode).

Monitor 114 may be configured to determine one or more physiological or other patient parameters based at least in part on information from one or more sensor units such as sensor unit 112. For example, monitor 114 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some examples, processing circuitry of system 110 may perform calculations on outputs from the sensor units or an intermediate device and the result of the calculations may be passed to monitor 114 if the processing circuitry is not included in monitor 114. Further, monitor 114 may include display 120 configured to display the physiological parameters or other information about the system.

Display 120 may be configured to display a DOA index score generated for a patient, where the DOA index score is indicative of the patient's DOA. In the example shown, monitor 114 may also include a speaker 122 configured to provide a sound that may be used in various other examples, such as for example, sounding an audible alarm in the event that a DOA index score determined by system 110 for a particular patient is above or below a predetermined threshold value or range of values. In some examples, physiological monitoring system 110 may include a stand-alone monitor in communication with the monitor 114 via a cable or a wireless network link.

In some examples, sensor unit 112 may be communicatively coupled to monitor 114 via a cable 124 at input or port 136. Cable 124 may include electrical conductors (e.g., wires for transmitting electrical signals from sensor unit 112), optical fibers (e.g., multi-mode or single-mode fibers for transmitting emitted light from sensor unit 112), any other suitable components, any suitable insulation or sheathing, or any combination thereof. In some examples, a wireless transmission device (not shown) or the like may be used instead of or in addition to cable 124. Monitor 114 may include a sensor interface configured to receive signals from sensor unit 112, provide signals and power to sensor unit 112, or otherwise communicate with sensor unit 112. The sensor interface may include any suitable hardware, software, or both, which may be allow communication between monitor 114 and sensor unit 112.

In the illustrated example, monitoring system 110 includes monitor 126. Although system 110 includes two monitors, other examples of system 110 may include only a single monitor or more than two monitors that are configured to perform the function attributed to monitor 114 and monitor 126. Monitor 126 may include any suitable display, such as, but not limited to, a cathode ray tube display, a flat panel display (as shown) such as a liquid crystal display (LCD) or a plasma display or may include any other type of suitable monitor configured, e.g., to display of a DOA index score or other information generated based on a determined DOA index score in accordance with examples of the disclosure. Monitor 126 may be configured to determine one or more physiological parameters and to present information from monitor 114 and/or other monitoring devices via a display 128. For example, monitor 126 may be configured to display information regarding a subject's DOA index score, and EEG, EMG, EOG, blood oxygen saturation (referred to as an “SpO₂” measurement), a blood pressure (BP), a heart rate (HR), body temperature, a facial expression signal (e.g., as determined based on an image captured by a camera used to monitor the subject), a facial color (e.g., as determined based on an image captured by a camera used to monitor the subject), concentration or partial pressure of carbon dioxide (e.g., in a patient's expiratory volume), and/or an auditory evoked potentials (AEP). Physiological monitor 126 may include a speaker 130, e.g., to present an auditory message to a user.

Monitor 114 may be communicatively coupled to monitor 126 via a cable 132 or 134 that is coupled to a sensor input port or a digital communications port, respectively and/or may communicate wirelessly (not shown). In addition, monitor 114 and/or monitor 126 may be coupled to a network to enable the sharing of information with servers or other workstations (not shown). Monitor 114 may be powered by a battery (not shown) or by a conventional power source such as a wall outlet.

As depicted in FIG. 1, monitor 126 may be communicably coupled to electrophysiological sensor 150. This coupling may occur through monitor interface cable 140, which connects to processing module 138, which itself connects to electrophysiological sensor 150 via physiological information cable 142. Processing module 138 includes processing circuitry that may perform any of a number of processing operations (e.g., those described below), and may be implemented as described herein with reference to monitor 114. For example, processing module 138 may be a BISx® module, which may be configured to identify characteristics of electrophysiological sensor 150 (e.g., sensor arrangement, usage history) and/or to deliver signals (in raw or processed form) from electrophysiological sensor 150 to multi-parameter physiological monitor 126. Electrophysiological sensor 150 may include one or more individual electrophysiological sensors (such as electrodes 144, 146, and 148), which may be positioned at one or more body sites on a subject, e.g., as external scalp electrodes. Although system 110 illustrates three electrodes (electrodes 144, 146, and 148), any suitable number of electrodes may be used (e.g., 10 to 20 electrodes).

In an example, monitor 126 may be configured to display a physiologically-based parameter, such as a DOA index score, based at least in part on a signal from electrophysiological sensor 150 over an interval of time and at a particular frequency, which may be adjusted by a user (e.g., the last 15 to 30 seconds, and updated every second). Additionally, or alternatively, monitor 126 may be configured to display a generated DOA index score, an EEG signal, EOG, signal, ECG, and/or EMG signal.

In some examples, electrophysiological sensor 150 may be connected directly to monitor 126, without the use of processing module 138. In an example, processing module 138 may be included within monitor 126 or within monitor 114. In an example, both sensor 112 and electrophysiological sensor 150 may be communicably coupled to common processing circuitry (e.g., processing module 138) which may transmit information based on signals from one or more of the sensors to a monitoring device (e.g., monitor 126). As described above, sensors 112 and 150 may be configured in a unitary sensor body or may be physically attached to each other. In an example, monitor 126 and monitor 114 may be combined into a single monitoring device. It will be noted that any suitable configuration of sensing and monitoring devices adapted to perform the techniques described herein may be used.

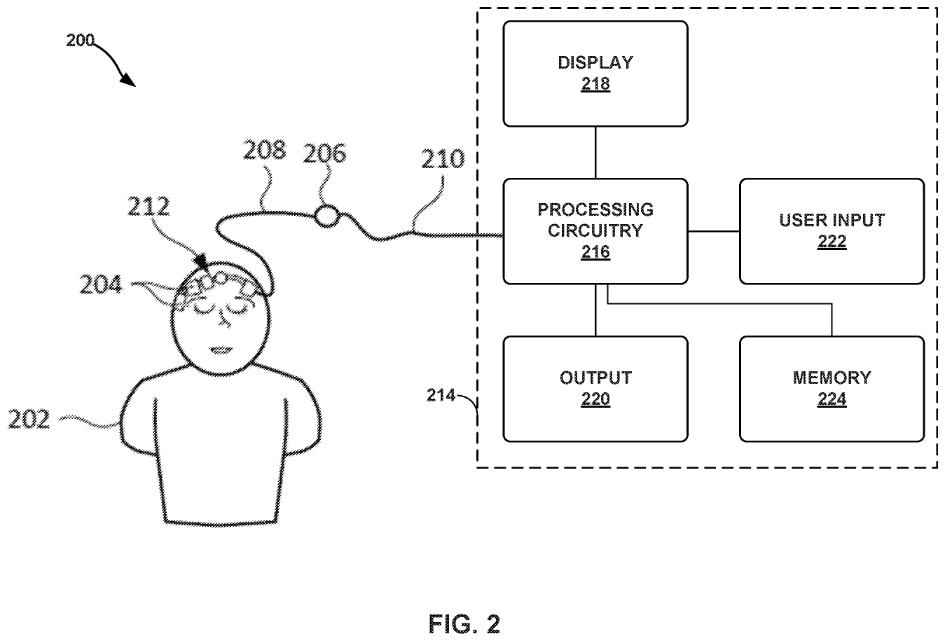

FIG. 2 is a block diagram of an example monitoring system 200 in accordance with some examples of the present disclosure. Monitoring system 200 includes sensor unit 212, including one or more surface (e.g., scalp) electrodes 204, which is communicatively coupled by cable 208 to processing module 206, which includes processing circuitry. Processing module 206 may be communicatively coupled by cable 210 to processing system 214. For example, cable 210 may be coupled to an input of processing system 214. In the illustrated example, processing system 214 may include processing circuitry 216 coupled to display 218, output 220, and user input 222.

In some examples, sensor unit 212 may include EEG leads electrically connected to the head of patient 202 by one or more surface electrodes 204, which, in some examples, are part of a BIS® 4 Electrode Sensor (Medtronic plc, Dublin, Ireland). In some examples, sensor unit 212 may detect electrical activity of a brain of subject 202 (e.g., to generate an EEG) and transmit an electrical signal indicative of the electrical activity over cable 208 to processing module 206, which may generate and transmit an input signal, including information based on signals from sensor unit 212, over cable 210 to processing system 214. The signals generated by sensor unit 212 may be applied to any device used to process EEG signals. For example, sensor unit 212 may be applied to a Bispectral Index (BIS®) generator of the type disclosed in U.S. Pat. No. 5,458,117, issued Oct. 17, 1995, the entirety of which is incorporated by reference herein. Additionally, or alternatively, the EEG signals generated by sensor unit 212 may be processed to generate a DOA index score in the manner described herein.

In some examples, processing module 206 may correspond to processing module 138 of FIG. 1. For example, processing module 206 may be a BISx® module, which may be configured to identify characteristics of sensor unit 212 (e.g., sensor arrangement, usage history) and/or to transmit an input signal over cable 210 to processing system 214. In some examples, the input signal may include signals (in raw or processed form) from sensor unit 212. The input signal may include, for example, an EEG, EOG, and/or EMG signal generated by one or more surface electrodes 204 of sensor unit 212. In some examples, processing module 206 may include an amplifier or other suitable signal EEG, EOG, and/or EMG processing components, and the input signal transmitted over cable 210 may include signals generated by one or more of these components. In some examples, the input signal may be representative of cerebral activity of subject 202, and processing system 214 may receive the input signal and determine a DOA index score indicative of the DOA of subject 202. In some examples, sensor unit 212 may be connected directly to processing system 214, without the use of processing module 206. In some examples, processing module 206 may be included within processing system 214. It will be understood that any suitable configuration of sensing and monitoring devices adapted to perform the techniques described herein may be used.

Processing circuitry 216 of processing system 214, as well as processing module 206 and other processing modules or circuitry described herein, may be any suitable software, firmware, hardware, or combination thereof. Processing circuitry 216 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 216, may be provided by processing circuitry of a hardware device, e.g., as supported by software and/or firmware.

In some examples, processing circuitry 216 is configured to determine physiological information associated with patient 202. For example, processing circuitry 216 may determine a DOA index score or any other suitable physiological parameter, such as those described herein. Processing circuitry 216 may perform any suitable signal processing of input signal 210 to filter the input signal, such as any suitable band-pass filtering, adaptive filtering, closed-loop filtering, any other suitable filtering, and/or any combination thereof. Processing circuitry 216 may also receive input signals from additional sources (not shown). For example, processing circuitry 216 may receive an input signal containing information about treatments provided to the patient. Additional input signals may be used by processing circuitry 216 in any of the calculations or operations it performs in accordance with processing system 200. In some examples, processing circuitry 216 may be adapted to execute software, which may include an operating system and one or more applications, as part of performing the functions described herein. In some examples, processing circuitry 216 may include one or more processing circuitry for performing each or any combination of the functions described herein.

In some examples, processing circuitry 216 may be coupled to memory 224. Memory 224 may include any volatile or non-volatile media, such as a random-access memory (RAM), read only memory (ROM), non-volatile RAM (NVRAM), electrically erasable programmable ROM (EEPROM), flash memory, and the like. Memory 224 may be a storage device or other non-transitory medium. Memory 224 may be used by processing circuitry 216 to, for example, store fiducial information or initialization information corresponding to physiological monitoring. In some examples, processing circuitry 216 may store physiological measurements or previously received data from input signal 210 in memory 224 for later retrieval. In some examples, processing circuitry 216 may store determined values, such as DOA index score, or any other calculated values, in memory 224 for later retrieval.

Processing circuitry 216 may be coupled to display 218, user input 222, and output 220. In some examples, display 218 may include one or more display devices (e.g., monitor, PDA, mobile phone, tablet computer, any other suitable display device, or any combination thereof). For example, display 218 may be configured to display physiological information and a DOA index score determined by monitoring system 200. In some examples, display 218 may correspond to display 120 or 128 of FIG. 1. In some examples, user input 222 is configured to receive input from a user, e.g., information about subject 202, such as age, weight, height, diagnosis, medications, treatments, and so forth. In some examples, display 218 may exhibit a list of values which may generally apply to subject 202, such as, for example, age ranges or medication families, which the user may select using user input 222.

User input 222 may include components for interaction with a user, such as a keypad and a display, which may be the same as display 218. In some examples, the display may be a cathode ray tube (CRT) display, a liquid crystal display (LCD) or light emitting diode (LED) display and the keypad may take the form of an alphanumeric keypad or a reduced set of keys associated with particular functions. User input 222, additionally or alternatively, include a peripheral pointing device, e.g., a mouse, via which a user may interact with the user interface. In some examples, the displays may include a touch screen display, and a user may interact with user input 222 via the touch screens of the displays. In some examples, the user may also interact with user input 222 remotely via a networked computing device.

In some examples, output 220 may include one or more medical devices (e.g., a medical monitor that displays various physiological or other parameters, a medical alarm, or any other suitable medical device that either displays physiological parameters or uses the output of processing circuitry 216 as an input), one or more audio devices, one or more printing devices, any other suitable output device, or any combination thereof. For example, output 220 may generate a printed output of physiological information or DOA index score determined by monitoring system 200. In some examples, output 220 may be part of monitor 126 or monitor 114.

In some examples, output 220 may include a communications interface that may enable processing system 214 to exchange information with external devices. The communications interface may include any suitable hardware, software, or both, which may enable monitoring system 200 (e.g., processing system 214) to communicate with electronic circuitry, a device, a network, or any combinations thereof. The communications interface may include one or more receivers, transmitters, transceivers, antennas, plug-in connectors, ports, communications buses, communications protocols, device identification protocols, any other suitable hardware or software, or any combination thereof. The communications interface may be configured to allow wired communication (e.g., using USB, RS-232, Ethernet, or other standards), wireless communication (e.g., using Wi-Fi, IR, WiMAX, BLUETOOTH, or other standards), or both. For example, the communications interface may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some examples, the communications interface may include an internal bus such as, for example, one or more slots for insertion of expansion cards.

Monitoring system 200 may be incorporated into monitoring system 110 of FIG. 1. For example, sensor unit 212 may be implemented as part of sensor unit 150. Processing system 214 may be implemented as part of monitor 114 or multi-parameter physiological monitor 126 of FIG. 1. Display 218 may be implemented as display 120 or 128 of FIG. 1. Furthermore, all or part of monitoring system 200 may be embedded in a small, compact object carried with or attached to subject 202 (e.g., a watch, other piece of jewelry, or a smart phone). In some examples, a wireless transceiver (not shown) may also be included in monitoring system 200 to enable wireless communication with other components of physiological monitoring system 110 of FIG. 1. As such, monitoring system 200 of FIG. 2 may be part of a fully portable and continuous subject monitoring solution. In some examples, a wireless transceiver (not shown) may also be included in monitoring system 200 to enable wireless communication with other components of monitoring system 110 of FIG. 1. For example, processing module 206 may communicate its generated input signal over BLUETOOTH, 802.11, WiFi, WiMax, cable, satellite, Infrared, or any other suitable transmission scheme. In some examples, a wireless transmission scheme may be used between any communicating components of monitoring system 200. In some examples, monitoring system 200 may include one or more communicatively coupled modules configured to perform particular tasks. In some examples, monitoring system 200 may be included as a module communicatively coupled to one or more other modules

The components of monitoring system 200 that are shown and described as separate components are shown and described as such for illustrative purposes only. In other examples the functionality of some of the components may be combined in a single component. For example, the functionality of processing circuitry 216 and processing module 206 may combined in a single processing circuitry system. Additionally, the functionality of some of the components shown and described herein may be divided over multiple components. Additionally, monitoring system 200 may perform the functionality of other components not show in FIG. 2. In some examples, the functionality of one or more of the components may not be required. In some examples, all of the components can be realized in processing circuitry.

In some examples, any of the processing components and/or circuits, or portions thereof, of FIGS. 1 and 2, including sensors 112, 150, and 212, monitors 114 and 126, processing circuitry 216, and processing system 214 may be referred to collectively as processing equipment. For example, processing equipment may be configured to amplify, filter, sample and digitize an input signal from sensors 112, 150, and 212 (e.g., using an analog-to-digital converter), determine physiological information and higher order statistical measures from the digitized signal, and display the physiological information. The processing equipment may include one or more processing circuitry. In some examples, all or some of the components of the processing equipment may be referred to as a processing module.

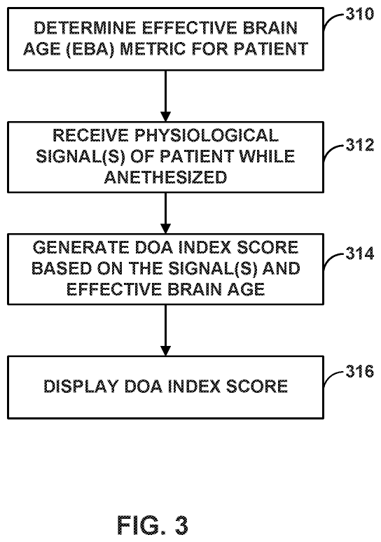

FIG. 3 is a flow diagram illustrating an example technique for generating a DOA index score indicative the depth of anesthesia of a patient. For purposes of description, the technique of FIG. 3 is described with regard to monitoring system 200. However, the example technique may be employed by any suitable system.

As shown in FIG. 3, monitoring system 200 may determine the effective brain age (EBA) metric of patient 202 (310). For example, processing circuitry 216 may determine the EBA metric of patient 202 based on one or more sensed brain signals (e.g., an EEG signal) of patient 202. Additionally, or alternatively, processing circuitry 216 may determine the EBA metric of patient 202 based on two or more patient parameters. Processing circuitry 216 may determine the EBA metric of patient 202 prior to patient 202 being anesthetized, e.g., during a pre-operation session, and/or while anesthetized, e.g., but prior to the generation of a DOA index score.

Example patient parameters may include at least one of a physiological parameter of the patient, a co-morbidity of the patient; a frailty of the patient, a baseline brain signal of the patient (e.g., a brain signal sensed before the patient is anesthetized), or patient medical history. In the case of a baseline brain signal, the baseline brain signal may be compared to population baseline when determining the EBA of a patient. Example physiological parameters may include patient brain signals (e.g., EEG, EMG, and/or EOG signals), patient biological age, patient heart rate and/or patient blood pressure. Processing circuitry 216 may receive input, e.g., from electrode(s) 204, user input 222, and/or sensor 212, which is indicative of the patient parameters. In some examples, co-morbidity and frailty of a patient may be determined based on assessments e.g., as described by Cardiovascular Health Study (CHS) (Fried, L. P., et al., Frailty in older adults: evidence for a phenotype. J Gerontol A Biol Sci Med Sci, 2001. 56(3): p. M146-56.)

Processing circuitry 216 may evaluate the received input and determine an EBA metric for patient 202 based on the input. For example, in the case of a brain signal (e.g., an EEG signal), the effective brain age of the patient may be determined by comparing to statistical characteristic of the age-group of several EEG driven parameters (e.g., beta band to delta band ratio, bands peak location). In some examples, processing circuitry 216 may evaluate the received input and determine an EBA metric for patient 202 based on the input to classify patient 202 within two or more EBA classes (e.g., two classes of “adult” and “old adult” with “old adult” corresponding to an EBA that is greater than “adult” or three classes of “adult” and “old adult” and “very old adult” corresponding to increasing EBAs in those classes).

In some examples, processing circuitry 216 may be configured to display the determined EBA for patient 202 via display 218. For example, the determined EBA for patient 202 may be displayed as a numerical value (e.g., on a scale of 1-10, with 1 being the lowest EBA and 10 being the highest EBA). Alternatively, or additionally, the EBA for patient 202 may be indicated via display of a non-numerical technique such as, e.g., using a color scale where different colors correspond to different relative levels of DOA (e.g., green reflecting a relatively low, yellow reflecting a medium EBA, and red reflecting a relatively high EBA) or text stating the level of EBA (e.g., “low EBA,” “medium EBA,” or “high EBA”). The display of the EBA may allow a clinician or other operator to verify or otherwise evaluate the determined EBA of patient 202 prior to using the EBA in determining the DOA index score of patient 202, as will be explained further below.

After determining the EBA for patient 202, processing circuitry 216 may receive physiological signals of patient 202 when patient 202 is anesthetized (312) and generate a DOA index score based on the received signals and determined EBA of patient 202 (314). For example, while patient 202 is under anesthesia, processing circuitry 216 may receive input signals, e.g., from electrode(s) 204, and/or sensor 112, 150, and/or 212 (312). Based on the received signals and the determined EBA of patient 202, processing circuitry 216 may generate a DOA index score indicative of the DOA for patient 202 using an algorithm that corresponds to the EBA for patient 202 (314). For example, processing circuitry 216 may analyze the received signals to identify characteristics of the signals that reflect the relative level of the DOA of patient 202 for a patient having the EBA determined for patient 202. Such characteristics may be predetermined and stored by memory 224. Example signal characteristics may include signal characteristics signal amplitude values or frequency domain characteristics, e.g., power level in a specific frequency band or ratio of power levels in different frequency bands. Processing circuitry 216 may then display an indication of the DOA index score, e.g., via display 218 (316). The technique of FIG. 3 may be performed preoperatively before a medical procedure (e.g., while patient 202 is anesthetized but before a surgeon begins operating on patient 202), during the medical procedure (e.g., while patient 202 is anesthetized during the procedure), and/or after the medical procedure (e.g., while patient 202 is anesthetized and in post-operation care and subsequently is no longer anesthetized).

In some examples, processing circuitry 216 may generate a DOA index score based on one or more sensed physiological parameters of patient 202, e.g., a DOA index score generated based on a single type of physiological parameter or more than one type of physiological parameters. For example, processing circuitry 216 may generate the DOA index score based on one or more of an electroencephalogram (EEG) signal, an electromyography (EMG) signal, an electrooculography (EOG) signal, a blood pressure (BP) signal, a heart rate (HR) signal, a temperature signal, a pulse oximeter (SpO₂) signal, a plethysmograph (finger and/or forehead) signal, a facial expression signal (e.g., as indicated by an image captured by a camera used to monitor patient 202), a facial color signal (e.g., as indicated by an image captured by a camera used to monitor patient 202), a capnogram signal, and/or an auditory evoked potentials (AEP).

In some examples, processing circuitry 216 may generate a DOA index score based on a first signal indicative of a first physiological parameter and a second signal indicative of a second physiological parameter, where the first signal comprises at least one of a blood pressure (BP) signal, a heart rate (HR) signal, a temperature signal, a pulse oximeter (SpO₂) signal, a plethysmograph (finger and/or forehead) signal, a facial expression signal (e.g., as received from camera used to monitor), a facial color signal (e.g., as indicated by an image captured by camera used to monitor patient 202), a capnogram signal, and/or an auditory evoked potentials (AEP). In some examples, the second signal may comprise a different patient parameter than the first signal, e.g., at least one of an electroencephalogram (EEG) signal, an electromyography (EMG) signal, an electrooculography (EOG) signal, a blood pressure (BP) signal, a heart rate (HR) signal, a temperature signal, a pulse oximeter (SpO₂) signal, a plethysmograph (finger and/or forehead) signal, a facial expression signal (e.g., as received from camera used to monitor), a facial color signal (as received from camera used to monitor), a capnogram signal, and/or an auditory evoked potentials (AEP).

As described above, processing circuitry 216 may generate the DOA index score based in part on the EBA determined for patient 202. In some examples, the particular physiological parameters analyzed by processing circuitry 216 to generate a DOA index score for patient 202 may be based on and vary depending on the EBA determined for patient 202. For example, for a relatively low EBA, processing circuitry 216 may analyze a first physiological parameter or first set of physiological parameters to determine the DOA index score for patient 202. The first physiological parameter or the first set of physiological parameters may be parameters that are indicative of the relative level of DOA in a patient with the relatively low EBA. Conversely, for a relatively high EBA, processing circuitry 216 may analyze a second physiological parameter or second set of physiological parameters to determined the DOA index score for patient 202. The second physiological parameter or the second set of physiological parameters may be parameters that are indicative of the relative level of DOA in a patient with the relatively high EBA. Additionally, or alternatively, processing circuitry 216 may generate the DOA index score by analyzing the same physiological parameter(s) for either the high or low EBA. However, processing circuitry 216 may identify different characteristics in the physiological parameters for a relatively low EBA versus a relatively high EBA as being indicative of the relative level of DOA of a patient. In some examples, the parameters for indication of a relatively older brain age may be chronological age, EEG pattern and, if available, a pre-op assessment.

In some examples, processing circuitry 216 may select a particular algorithm to analyze the received physiological signal(s) of patient 202 and generate the DOA index score in order to generate the DOA index score (314). Processing circuitry 216 may select the particular algorithm based on the EBA determined for patient 202 (310). For example, for a relatively low EBA, processing circuitry 216 may select a first algorithm to analyze the received physiological signal(s) of patient 202 and generate the DOA index score, and for a relatively high EBA, processing circuitry 216 may select a second algorithm different from the first algorithm to analyze the received physiological signal(s) of patient 202 and generate the DOA index score. Similar to that described above, using the first algorithm, processing circuitry 216 may analyze a first physiological parameter or first set of physiological parameters to determine the DOA index score of patient 202. Conversely, using the second algorithm, processing circuitry 216 may analyze a second physiological parameter or second set of physiological parameters to determine the DOA index score of patient 202. Additionally, or alternatively, processing circuitry 216 may analyze the same parameter(s) using the first and second algorithms but may identify a different characteristic in the parameter(s) as being indicative of a relative level of DOA of patient 202 when generating the DOA index score. Similarly, processing circuitry 216 may analyze the same parameter(s) using the first and second algorithms to identify for the same characteristic in the parameter(s), but with the characteristic being indicative of a different relative level of DOA of patient 202 based on the determined EBA when generating the DOA index score.

In some examples, rather than selecting different algorithms from a plurality of predetermined algorithms for different levels of EBA, processing circuitry 216 may be configured to adapt a baseline algorithm based on the determined EBA for patient 202. For example, processing circuitry 216 may modify the baseline algorithm to weigh one or more physiological parameters and/or characteristics of physiological parameters more or less as indicators of a relative level of DOA for patient 202 when generating a DOA index score using the adapted algorithm. Different adaptations to the baseline algorithm may be associated with different relative levels of EBA.

In some examples, processing circuitry 216 may present via display 218 the generated DOA index score (316), e.g., as a numerical value selected from a numerical scale, e.g., on a scale of 1 to 100 where 1 indicates the no DOA or the lowest DOA and where 100 indicates the highest DOA for a patient. Alternatively, or additionally, processing circuitry 216 may present the DOA index score for the patient via display of a non-numerical technique such as, e.g., using a color scale where different colors correspond to different relative levels of DOA (e.g., green reflecting a desired DOA, and red reflecting an undesirable DOA) or text stating the level of DOA (e.g., “low DOA,” “medium DOA,” or “high DOA”).

In some examples, processing circuitry 216 may receive the signal indicative of a patient physiological parameter (310) by at least receiving an electrical physiological signal of patient, e.g., via electrode(s) 204 and/or sensor 112, 150, and 212. For example, processing circuitry 216 may receive EEG, EMG, ECG, and/or EOG signals from electrodes 204 and/or other sensor. Processing circuitry 216 may analyze the received EEG, EMG, ECG, and/or EOG signals to identify one or more characteristics of the received electrical physiological signals that are indicative of a patient DOA. The characteristics of the received electrical physiological signals that are indicative of DOA may be identified using any suitable technique including, e.g., analyzing the same type of received signals from prior patient.

As described herein, the characteristics of the received electrical physiological signals that are indicative of DOA may differ based on the particular EBA determined for patient 202. For example, processing circuitry 216 may identify a first characteristic or first set of characteristics of the received electrical physiological signals to determine the relative level of DOA for patient 202 when patient 202 is determined to have a relatively low EBA. Conversely, processing circuitry 216 may identify a second characteristic or second set of characteristics of the received electrical physiological signals (different from the first characteristic or first set of characteristics of the received electrical physiological signals) to determine the relative level of DOA for patient 202 when patient 202 is determined to have a relatively high EBA.

In some examples, the or more characteristics of the received electrical physiological signals that are indicative of the relative DOA of patient 202 include at least one of an amplitude of the EEG signal, a K-complex of the EEG signal, or a suppression of the EEG signal. Thus, in some examples, processing circuitry 216 may receive an EEG signal (310) and analyze the EEG signal to determine at least one of an amplitude of the EEG signal, a K-complex of the EEG signal, or a suppression of the EEG signal. For example, processing circuitry 216 may identify an amplitude of the EEG signal (e.g., compared to a predetermined baseline, range, or threshold amplitude) that is characteristic of a relatively low, medium, or high DOA of patient 202.

In some examples, processing circuitry 216 may analyzed the received EEG signals within one or more specific frequency bands to identify one or more frequency domain characteristics, e.g., power, that indicate the relative DOA of patient 202. One example of the frequency bands is shown in Table 1:

TABLE 1
Frequency bands

| Frequency (f) Band | |
|---|---|
| Hertz (Hz) | Frequency Information |
| f < 5 Hz | δ (delta frequency band) |
| 5 Hz ≤ f ≤ 10 Hz | α (alpha frequency band) |
| 10 Hz ≤ f ≤ 30 Hz | β (beta frequency band) |
| 50 Hz ≤ f ≤ 100 Hz | γ (gamma frequency band) |
| 100 Hz ≤ f ≤ 200 Hz | high γ (high gamma frequency band) |

The frequency ranges for the frequency bands shown in Table 1 are merely examples. The frequency ranges may differ in other examples. For example, another example of frequency ranges for frequency bands are shown in Table 2:

TABLE 2
Frequency bands

| Frequency (f) Band | |
|---|---|
| Hertz (Hz) | Frequency Information |
| f < 5 Hz | δ (delta frequency band) |
| 5 Hz ≤ f ≤ 8 Hz | q (theta frequency band) |
| 8 Hz ≤ f ≤12 Hz | α (alpha frequency band) |
| 12 Hz ≤ f ≤16 Hz | s (sigma or low beta frequency band) |
| 16 Hz ≤ f ≤ 30 Hz | High β (high beta frequency band) |
| 50 Hz ≤ f ≤ 100 Hz | γ (gamma frequency band) |
| 100 Hz ≤ f ≤ 200 Hz | high γ (high gamma frequency band) |

In some examples, processing circuitry 216 may receive an EEG signal from the right hemisphere of the brain of patient 202 and also an EEG signal from the left hemisphere of the brain of patient 202. Processing circuitry 216 may then compare the EEG signal from the right hemisphere to the EEG signal from the left hemisphere to identify characteristic(s) between the two signals that are indicative of the relative level of DOA for patient 202. For example, processing circuitry 216 may compare the overall power in each hemisphere and/or power within in specific frequency bands of the EEG signal in each hemisphere to identify characteristics that are indicative of a relatively low DOA, relatively medium DOA, or relatively high DOA of patient 202. For example, processing circuitry may identify a ratio of power in a specific frequency band between each hemisphere that indicates relatively low, medium, or high DOA of patient 202. As another example, processing circuitry 216 may determine the power level in a delta frequency band of an EEG signal and compare the power level to a predetermined threshold value to determine the DOA index score for patient 202.

In some examples, processing circuitry 216 may analyze the bispectrum of the received signal, e.g., as reflected in a BIS® index. A BIS® index may be a processed parameter which may be derived utilizing a composite of measures from the EEG and physiological signal processing techniques including bispectral analysis, power spectral analysis, and time domain analysis. The BIS algorithm may be based at least in part on EEG signal features (bispectral and others) which may be highly correlated with sedation and/or hypnosis, including the degree of high frequency (e.g., 14 Hz to 30 Hz) activation, the amount of low frequency synchronization, the presence of nearly suppressed periods within the EEG, and the presence of fully suppressed (i.e., isoelectric, “flat line”) periods within an EEG. The BIS index may provide an indication of a subject's depth of consciousness, with an index value of 0 representing a “flat line” EEG and an index value of 100 indicating a fully awake subject. Examples of systems configured to generate a Bispectral Index (BIS®) include generators of the type disclosed in U.S. Pat. No. 5,458,117, issued Oct. 17, 1995. Processing circuitry 216 may identify a pattern or other characteristic of the BIS or other bispectrum parameter that indicates a relatively DOA of patient 202.

In some examples, the EEG or other signals received by processing circuitry 216 may be recorded when patient 202 is anesthetized (e.g., during the medical procedure). In some examples, the EEG or other signals received by processing circuitry 216 may be recorded when patient 202 is responding to stimuli (e.g., audible evoked stimuli) as baseline or threshold value that is used for comparison when patient 202 is anesthetized. Such signals may be analyzed alone or in comparison to EEG or other signals in which patient 202 is not responding to such stimuli.

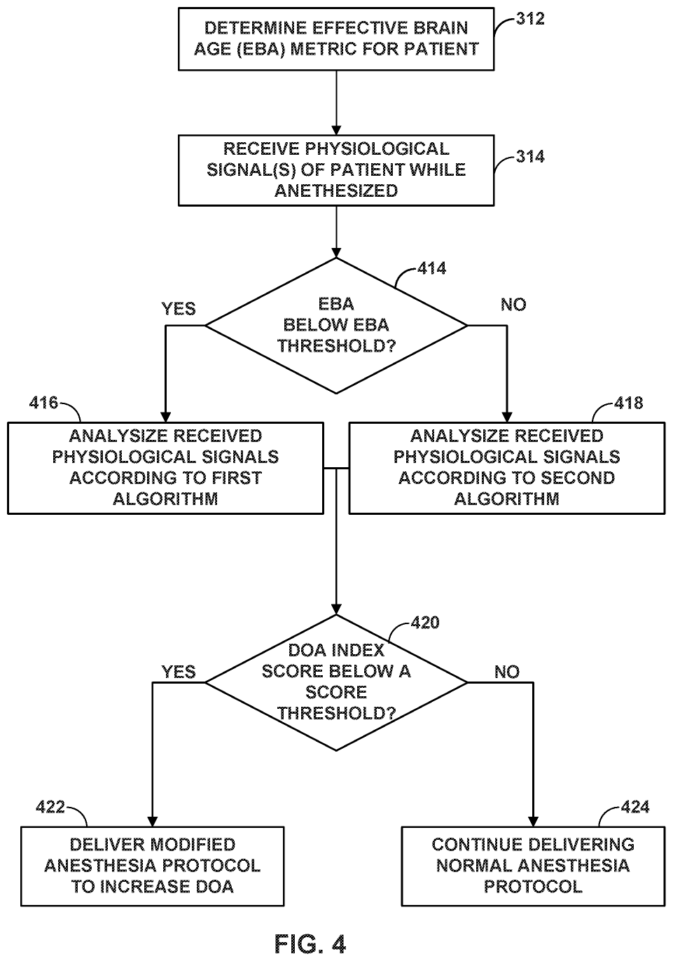

FIG. 4 is a flow diagram illustrating an example technique for generating a DOA index score indicative the depth of anesthesia of a patient. For purposes of description, the technique of FIG. 4 is described with regard to monitoring system 200. However, the example technique may be employed by any suitable system. The example technique of FIG. 4 may be carried out while patient 202 is anesthetized for a medical procedure.

Similar to that described for the example technique of FIG. 3, processing circuitry 216 may initially determine an EBA for patient 202 (312), e.g., prior to anesthetization. While patient is under anesthesia, processing circuitry 216 may then receive input signals, e.g., from electrode(s) 204 and/or sensor 112, 150, and 212, indicative of one or more physiological parameters of patient 202 (314).

Processing circuitry 216 may then select an algorithm to employ when analyzing the received signals to generate a DOA index score for patient 202. For example, as shown in FIG. 4, processing circuitry 216 may determine whether the EBA determined for patient 202 is less than a predetermined EBA threshold (414), e.g., stored in memory 224. If the EBA determined for patient 202 is less than an EBA threshold, then processing circuitry 216 may analyze the received physiological signal(s) according to a first algorithm (416). Conversely, if the EBA determined for patient 202 is not less than the threshold, then processing circuitry 216 may analyze the received physiological signal(s) according to a second algorithm different from the first algorithm (416).

The first algorithm may be configured to generate an accurate DOA index score for patient 202 for a patient having a determined EBA less than the EBA threshold, e.g., using one or more physiological parameters and/or characteristics of one or more physiological parameters that are indicative of the DOA of patient 202. The second algorithm may be different than the first algorithm in that is may be configured to generate an accurate DOA index score for patient 202 for a patient having a determined EBA not less than the EBA threshold.

In either instance, based on the received signals, processing circuitry 216 may generate a DOA index score indicative of the DOA of patient 202 based on the determined EBA (312). As shown in FIG. 4, processing circuitry 216 may then determine whether or not the determined DOA index score is below a predetermined threshold (420), e.g., stored in memory 224. If the determined risk index score is below the threshold, then processing circuitry 216 may control a medical device to deliver a modified anesthesia protocol that is configured to increase the DOA of patient 202 (422). Additionally or alternatively, if the determined risk index score is below the threshold, then processing circuitry 210 may present (e.g., via display 218, via an auditory alert, via a haptic alert, or any combination thereof) a notification to the clinician that indicate a relative low DOA for patient 202, and the clinician may choose to modify the anesthetization protocol or take other steps to account for the low DOA index score and, e.g., try to increase the DOA index score. Conversely, if the determined DOA index score is not below the threshold level, then processing circuitry 210 may continue to deliver the anesthesia protocol without modification (424).

Although not shown in FIG. 4, in some examples, processing circuitry 216 may also determine if the determined DOA index score is above a predetermined upper threshold. If the DOA index score is above the upper threshold, then processing circuitry 210 may control a medical device to deliver a modified anesthesia protocol that is configured to decrease the DOA of patient 202. Additionally or alternatively, if the determined DOA index score is above the upper threshold, then processing circuitry 210 may present (e.g., via display 218, via an auditory alert, via a haptic alert, or any combination thereof) any notifications to the clinician that indicate a relative high DOA for patient 202, and the clinician may choose to modify the anesthetization protocol or take other steps to account for the high DOA index score.

In some examples, processing circuitry 216 may be configured to automatically modify the anesthesia protocol based on the determined DOA index score being above or below the threshold levels while in other examples a clinician and/or other medical personnel may initiate or approve the modified protocol, e.g., after being prompted by processing circuitry 216, e.g., via display 218. The threshold levels may be predetermined levels, e.g., set by a clinician or other medical personnel associated with the medical procedure of patient 202. Processing circuitry 216 may continuously or periodically (e.g., about once per second) determine the DOA index score for patient 202 over a period of time while patient 202 is anesthetized.

Any suitable technical methodologies may be used to generate a DOA index score based on inputs such as those described herein. Examples may include support vector machine (SVM), fuzzy logic, artificial neural networks, and the like. In some examples, state-of-the-art signal processing techniques such as wavelets and/or blind source separation, along with advanced machine learning techniques including artificial neural networks, random forests, and/or the like may be employed to determine a DOA index score for a patient, e.g., according to the example technique described herein.

Various aspects of the techniques may be implemented within one or more processers, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processors” may generally refer to any of the foregoing logic circuitry, alone or in combination with other logic circuitry or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more digital signal processors (DSPs), application-specific integrated circuits (ASICs), field-programmable gate arrays (FPGAs), general purpose microprocessor circuitry, or other equivalent integrated or discrete logic circuitry. Accordingly, the terms “processor” or “processors” as used herein may refer to one or more of any of the foregoing structures or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various aspects of the disclosure have been described. These and other aspects are within the scope of the following clauses and claims.

Clause 1. A method comprising determining, via processing circuitry, an effective brain age metric for a patient based on two or more patient parameters; receiving a signal indicative of a physiological parameter of the patient; and generating, via the processing circuitry, an index score indicative of depth of anesthesia (DOA) of the patient based on the received signal and the determined effective brain age metric for the patient.

Clause 2. The method of clause 1, wherein the two or more patient parameters include at least one of a physiological parameter of the patient, a co-morbidity of the patient, a frailty of the patient, a baseline brain signal of the patient, or patient medical history.

Clause 3. The method of clause 2, wherein the physiological parameter of the patient comprises at least one of a brain signal, a heart rate, a blood pressure, or a biological age.

Clause 4. The method of clause 2, wherein the baseline brain signal of the patient comprises at least one of a baseline electroencephalogram (EEG) signal, a baseline electromyography (EMG) signal, or a baseline electrooculography (EOG) signal.

Clause 5. The method of any of clauses 1-4, wherein the received signal comprises at least one of an electroencephalogram (EEG) signal, an electromyography (EMG) signal, an electrooculography (EOG) signal, a heart rate signal, or a blood pressure signal.

Clause 6. The method of any of clauses 1-5, wherein generating, via the processing circuitry, an index score indicative of DOA of the patient based on the received signal and the determined effective brain age metric for the patient comprises: determining that the effective brain age metric is greater than or equal to a threshold value, selecting an algorithm from a plurality of algorithms based on the determination that the effective brain age metric is greater than or equal to the threshold value, and generating the index score indicative of DOA of the patient based on the received signal using the selected algorithm.

Clause 7. The method of any of clauses 1-5, wherein generating, via the processing circuitry, an index score indicative of DOA of the patient based on the received signal and the determined effective brain age metric for the patient comprises: adapting an algorithm for determining the index score indicative of the DOA of the patient based on the determined effective brain age metric, and generating the index score indicative of DOA of the patient based on the received signal using the adaptive algorithm.

Clause 8. The method of any of clauses 1-7, further comprising presenting, via a display, the index score.

Clause 9. The method of any of clauses 1-8, wherein the received signal comprises an electroencephalogram (EEG) signal.

Clause 10. The method of any of clauses 1-9, further comprising sensing the received signal while the patient is anesthetized.

Clause 11. The method of any of clauses 1-10, wherein determining the effective brain age metric for the patient based on the two or more patient parameters comprises determining the effective brain age metric for the patient prior to the patient being anesthetized.

Clause 12. The method of any of clauses 1-11, further comprising receiving one or more signals indicative of the two or more patient parameters prior to the patient being anesthetized.

Clause 13. The method of any of clauses 1-12, wherein generating the index score comprises generating the index score while the patient is anesthetized.

Clause 14. The method of any of clauses 1 to 13, further comprising anesthetizing the patient.

Clause 15. The method of clause 14, further comprising modifying the anesthetization of the patient based on the generated index score.

Clause 16. The method of clause 15, further comprising determining that the generated index score is above an upper threshold value or below a lower threshold value, wherein modifying the anesthetization of the patient based on the generated index score comprises modifying the anesthetization of the patient based on the determination that the generated index score is above the upper threshold value or below the lower threshold value.

Clause 17. The method of any of clauses 1 to 16, wherein the index score comprises a numerical value indicative of the DOA for the patient.

Clause 18. A system comprising processing circuitry configured to perform the method of any of clauses 1 to 17.

Clause 19. A method comprising: determining, via processing circuitry, an effective brain age metric for a patient based on at least one brain signal of the patient; receiving a signal indicative of a physiological parameter of the patient; and generating, via the processing circuitry, an index score indicative of depth of anesthesia (DOA) of the patient based on the received signal and the determined effective brain age metric for the patient.

Clause 20. The method of clause 19, wherein the at least one brain signal of the patient includes at least one of a baseline electroencephalogram (EEG) signal, a baseline electromyography (EMG) signal, or a baseline electrooculography (EOG) signal.

Clause 21. The method of clause 19, wherein determining the effective brain age metric for the patient based on at least one brain signal of the patient comprises determining the effective brain age metric for the patient based on the at least one brain signal of the patient and one or more additional patient parameters.

Clause 22. The method of clause 21, wherein the one or more additional patient parameters includes at least one of a heart rate of the patient, a blood pressure of the patient, a biological age of the patient, a co-morbidity of the patient, a frailty of the patient, a baseline brain signal of the patient, or patient medical history.

Clause 23. The method of clause 22, wherein the baseline brain signal of the patient comprises at least one of a baseline electroencephalogram (EEG) signal, a baseline electromyography (EMG) signal, or a baseline electrooculography (EOG) signal.

Clause 24. The method of any of clauses 19-23, wherein the received signal comprises at least one of an electroencephalogram (EEG) signal, an electromyography (EMG) signal, an electrooculography (EOG) signal, a heart rate signal, or a blood pressure signal.

Clause 25. The method of any of clauses 19-24, wherein generating, via the processing circuitry, an index score indicative of DOA of the patient based on the received signal and the determined effective brain age metric for the patient comprises: determining that the effective brain age metric is greater than or equal to a threshold value, selecting an algorithm from a plurality of algorithms based on the determination that the effective brain age metric is greater than or equal to the threshold value, and generating the index score indicative of DOA of the patient based on the received signal using the selected algorithm.

Clause 26. The method of any of clauses 19-24, wherein generating, via the processing circuitry, an index score indicative of DOA of the patient based on the received signal and the determined effective brain age metric for the patient comprises: adapting an algorithm for determining the index score indicative of the DOA of the patient based on the determined effective brain age metric, and generating the index score indicative of DOA of the patient based on the received signal using the adaptive algorithm.

Clause 27. The method of any of clauses 19-26, further comprising presenting, via a display, the index score.

Clause 28. The method of any of clauses 19-27, wherein the received signal comprises an electroencephalogram (EEG) signal.

Clause 29. The method of any of clauses 19-28, further comprising sensing the received signal while the patient is anesthetized.

Clause 30. The method of any of clauses 19-29, wherein determining the effective brain age metric for the patient based on the at least one brain signal of the patient comprises determining the effective brain age metric for the patient prior to the patient being anesthetized.

Clause 31. The method of any of clauses 19-30, further comprising receiving one or more signals indicative of the at least one brain signal prior to the patient being anesthetized.

Clause 32. The method of any of clauses 19-31, wherein generating the index score comprises generating the index score while the patient is anesthetized.

Clause 33. The method of any of clauses 19-32, further comprising anesthetizing the patient.

Clause 34. The method of clause 33, further comprising modifying the anesthetization of the patient based on the generated index score.

Clause 35. The method of clause 34, further comprising determining that the generated index score is above an upper threshold value or below a lower threshold value, wherein modifying the anesthetization of the patient based on the generated index score comprises modifying the anesthetization of the patient based on the determination that the generated index score is above the upper threshold value or below the lower threshold value.

Clause 36. The method of any of clauses 19 to 35, wherein the index score comprises a numerical value indicative of the DOA for the patient.

Clause 37. A system comprising processing circuitry configured to perform the method of any of clauses 19-36.

Clause 38. A system comprising: processing circuitry configured to: determine an effective brain age metric for a patient based on two or more of patient parameters; receive a signal indicative of a physiological parameter of the patient; and generate, via the processing circuitry, an index score indicative of depth of anesthesia (DOA) of the patient based on the received signal and the determined effective brain age metric for the patient.

Clause 39. The system of clause 38, wherein the two or more patient parameters include at least one of a physiological parameter of the patient, a co-morbidity of the patient, a frailty of the patient, a baseline brain signal of the patient, or patient medical history.

Clause 40. The system of clause 39, wherein the physiological parameter of the patient comprises at least one of a brain signal, a heart rate, a blood pressure, or a biological age.

Clause 41. The system of clause 39, wherein the baseline brain signal of the patient comprises at least one of a baseline electroencephalogram (EEG) signal, a baseline electromyography (EMG) signal, or a baseline electrooculography (EOG) signal.

Clause 42. The system of any of clauses 38-41, wherein the received signal comprises at least one of an electroencephalogram (EEG) signal, an electromyography (EMG) signal, an electrooculography (EOG) signal, a heart rate signal, or a blood pressure signal.

Clause 43. The system of any of clauses 38-42, wherein the processing circuitry is configured to: determining that the effective brain age metric is greater than or equal to a threshold value, selecting an algorithm from a plurality of algorithms based on the determination that the effective brain age metric is greater than or equal to the threshold value, and generating the index score indicative of DOA of the patient based on the received signal using the selected algorithm.

Clause 44. The system of any of clauses 38-43, wherein the processing circuitry is configured to: adapting an algorithm for determining the index score indicative of the DOA of the patient based on the determined effective brain age metric, and generating the index score indicative of DOA of the patient based on the received signal using the adaptive algorithm.

Clause 45. The system of any of clauses 38-44, further comprising a display configured to present, via the display, the index score.

Clause 46. The system of any of clauses 38-45, wherein the received signal comprises an electroencephalogram (EEG) signal.

Clause 47. The system of any of clauses 38-46, where the received signal is sensed while the patient is anesthetized.

Clause 48. The system of any of clauses 38-47, wherein the processing circuitry is configured to determine the effective brain age metric for the patient prior to the patient being anesthetized.

Clause 49. The system of any of clauses 38-48, wherein the processing circuitry is configured to receive one or more signals indicative of the two or more patient parameters prior to the patient being anesthetized.

Clause 50. The system of any of clauses 38-49, wherein the processing circuitry is configured to generate the index score while the patient is anesthetized.

Clause 51. The system of any of clauses 38-50, wherein the processing circuitry is configured to control delivery of an anesthetization therapy configured to anesthetize the patient.

Clause 52. The system of clause 51, wherein the processing circuitry is configured to modify the delivery of the anesthetization therapy based on the generated index score.

Clause 53. The system of clause 52, wherein the processing circuitry is configured to determine that the generated index score is above an upper threshold value or below a lower threshold value, and modify the delivery of the anesthetization therapy of the patient based on the determination that the generated index score is above the upper threshold value or below the lower threshold value.

Clause 54. The system of any of clauses 38-53, wherein the index score comprises a numerical value indicative of the DOA for the patient.

Clause 55. A system comprising: processing circuitry configured to: determining, via processing circuitry, an effective brain age metric for a patient based on at least one brain signal of the patient; receiving a signal indicative of a physiological parameter of the patient; and generating, via the processing circuitry, an index score indicative of depth of anesthesia (DOA) of the patient based on the received signal and the determined effective brain age metric for the patient.

Clause 56. The system of clause 55, wherein the at least one brain signal of the patient includes at least one of a baseline electroencephalogram (EEG) signal, a baseline electromyography (EMG) signal, or a baseline electrooculography (EOG) signal.

Clause 57. The system of clause 55, wherein the processing circuitry is configured to determine the effective brain age metric for the patient based on the at least one brain signal of the patient and one or more additional patient parameters.

Clause 58. The system of clause 57, wherein the one or more additional patient parameters includes at least one of a heart rate of the patient, a blood pressure of the patient, a biological age of the patient, a co-morbidity of the patient, a frailty of the patient, a baseline brain signal of the patient, or patient medical history.

Clause 59. The system of clause 58, wherein the baseline brain signal of the patient comprises at least one of a baseline electroencephalogram (EEG) signal, a baseline electromyography (EMG) signal, or a baseline electrooculography (EOG) signal.

Clause 60. The system of any of clauses 55-59, wherein the received signal comprises at least one of an electroencephalogram (EEG) signal, an electromyography (EMG) signal, an electrooculography (EOG) signal, a heart rate signal, or a blood pressure signal.

Clause 61. The system of any of clauses 55-60, wherein the processing circuitry is configured to: determine that the effective brain age metric is greater than or equal to a threshold value, select an algorithm from a plurality of algorithms based on the determination that the effective brain age metric is greater than or equal to the threshold value, and generate the index score indicative of DOA of the patient based on the received signal using the selected algorithm.

Clause 62. The system of any of clauses 55-60, wherein the processing circuitry is configured to: adapt an algorithm for determining the index score indicative of the DOA of the patient based on the determined effective brain age metric, and generate the index score indicative of DOA of the patient based on the received signal using the adaptive algorithm.

Clause 63. The system of any of clauses 55-62, further comprising a display configured to present, via the display, the index score.

Clause 64. The system of any of clauses 55-63, wherein the received signal comprises an electroencephalogram (EEG) signal.

Clause 65. The system of any of clauses 55-64, wherein the processing circuitry is configured to sense the received signal while the patient is anesthetized.

Clause 66. The system of any of clauses 55-65, wherein the processing circuitry is configured to determine the effective brain age metric for the patient prior to the patient being anesthetized.

Clause 67. The system of any of clauses 55-66, wherein the processing circuitry is configured to receive one or more signals indicative of the at least one brain signal prior to the patient being anesthetized.

Clause 68. The system of any of clauses 55-67, wherein the processing circuitry is configured to generate the index score while the patient is anesthetized.

Clause 69. The system of any of clauses 55-68, wherein the processing circuitry is configured to control delivery of an anesthetization therapy configured to anesthetize the patient.

Clause 70. The system of clause 69, wherein the processing circuitry is configured to modify the delivery of the anesthetization therapy based on the generated index score.

Clause 71. The system of clause 70, wherein the processing circuitry is configured to determine that the generated index score is above an upper threshold value or below a lower threshold value, and modify the anesthetization therapy delivery to the patient based on the determination that the generated index score is above the upper threshold value or below the lower threshold value.

Clause 72. The system of any of clauses 55 to 71, wherein the index score comprises a numerical value indicative of the DOA for the patient.

What is claimed is:

1. A method comprising:
determining, via processing circuitry, an effective brain age metric for a patient based on two or more patient parameters;
receiving a signal indicative of a physiological parameter of the patient; and
generating, via the processing circuitry, an index score indicative of depth of anesthesia (DOA) of the patient based on the received signal and the determined effective brain age metric for the patient.

2. The method of claim 1, wherein the two or more patient parameters include at least one of a physiological parameter of the patient, a co-morbidity of the patient, a frailty of the patient, a baseline brain signal of the patient, or patient medical history.

3. The method of claim 2, wherein the physiological parameter of the patient comprises at least one of a brain signal, a heart rate, a blood pressure, or a biological age.

4. The method of claim 2, wherein the baseline brain signal of the patient comprises at least one of a baseline electroencephalogram (EEG) signal, a baseline electromyography (EMG) signal, or a baseline electrooculography (EOG) signal.

5. The method of claim 1, wherein the received signal comprises at least one of an electroencephalogram (EEG) signal, an electromyography (EMG) signal, an electrooculography (EOG) signal, a heart rate signal, or a blood pressure signal.

6. The method of claim 1, wherein generating, via the processing circuitry, an index score indicative of DOA of the patient based on the received signal and the determined effective brain age metric for the patient comprises:
determining that the effective brain age metric is greater than or equal to a threshold value;
selecting an algorithm from a plurality of algorithms based on the determination that the effective brain age metric is greater than or equal to the threshold value; and
generating the index score indicative of DOA of the patient based on the received signal using the selected algorithm.

7. The method of claim 1, wherein generating, via the processing circuitry, an index score indicative of DOA of the patient based on the received signal and the determined effective brain age metric for the patient comprises:
adapting an algorithm for determining the index score indicative of the DOA of the patient based on the determined effective brain age metric; and
generating the index score indicative of DOA of the patient based on the received signal using the adaptive algorithm.

8. The method of claim 1, further comprising presenting, via a display, the index score.

9. The method of claim 1, wherein the received signal comprises an electroencephalogram (EEG) signal.

10. The method of claim 1, further comprising sensing the received signal while the patient is anesthetized.

11. A system comprising processing circuitry configured to:
determine an effective brain age metric for a patient based on two or more of patient parameters;
receive a signal indicative of a physiological parameter of the patient; and
generate an index score indicative of depth of anesthesia (DOA) of the patient based on the received signal and the determined effective brain age metric for the patient.

12. The system of claim 11, wherein the two or more patient parameters include at least one of a physiological parameter of the patient, a co-morbidity of the patient, a frailty of the patient, a baseline brain signal of the patient, or patient medical history.

13. The system of claim 12, wherein the physiological parameter of the patient comprises at least one of a brain signal, a heart rate, a blood pressure, or a biological age.

14. The system of claim 12, wherein the baseline brain signal of the patient comprises at least one of a baseline electroencephalogram (EEG) signal, a baseline electromyography (EMG) signal, or a baseline electrooculography (EOG) signal.

15. The system of claim 11, wherein the received signal comprises at least one of an electroencephalogram (EEG) signal, an electromyography (EMG) signal, an electrooculography (EOG) signal, a heart rate signal, or a blood pressure signal.

16. The system of claim 11, wherein the processing circuitry is configured to:
determine that the effective brain age metric is greater than or equal to a threshold value,
select an algorithm from a plurality of algorithms based on the determination that the effective brain age metric is greater than or equal to the threshold value, and
generate the index score indicative of DOA of the patient based on the received signal using the selected algorithm.

17. The system of claim 11, wherein the processing circuitry is configured to:
adapt an algorithm for determining the index score indicative of the DOA of the patient based on the determined effective brain age metric, and
generate the index score indicative of DOA of the patient based on the received signal using the adaptive algorithm.

18. The system of claim 11, further comprising a display configured to present the index score.

19. The system of claim 11, wherein the received signal comprises an electroencephalogram (EEG) signal.

20. A method comprising:
determining, via processing circuitry, an effective brain age metric for a patient based on at least one brain signal of the patient;
receiving a signal indicative of a physiological parameter of the patient; and
generating, via the processing circuitry, an index score indicative of depth of anesthesia (DOA) of the patient based on the received signal and the determined effective brain age metric for the patient.
